(19) United States
(12) Patent Application Publication
(10) Pub. No.: US 2006/0167207 A1
(43) Pub. Date: Jul. 27, 2006

(54) Mixtures containing isocyanate stabilisers of the hindered phenol type with a high molecular weight

(75) Inventors: Kai Thiele, Schwarzheide (DE); Hauke Malz, Diepholz (DE); Thomas Flug, Wagenfeld (DE); Sylvia Rybicki, Ostercappeln (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: Basf Aktiengesellschaft, Ludwigshafen (DE)

(21) Appl. No.: 10/560,041

(22) PCT Filed: Jun. 8, 2004
(86) PCT No.: PCT/EP04/06159

§ 371 Date: Dec. 8, 2005

(30) Foreign Application Priority Data
Jun. 12, 2003 (DE) 10327009.4

(51) Int. Cl. C08G 18/16; C07C 265/02

(52) U.S. Cl. 528/48; 560/333

(57) ABSTRACT

The invention relates to a mixture (1) comprising (a) isocyanate and (b) stabilizers with a molar mass of from 600 to 10000 g/mol comprising at least two phenolic groups.

The invention relates to a mixture (1) comprising (a) isocyanate and (b) stabilizers, preferably amorphous or liquid stabilizers, with a molar mass of from 600 to 10000 g/mol, preferably from 700 to 3000 g/mol, comprising at least two phenolic groups. The invention further relates to a process for preparing polyurethanes, by using these mixtures as isocyanate component.

Because polyurethanes are versatile, they are some of the most important plastics in quantitative terms. Typical polyurethane applications are flexible foam applications, such as mattresses, foam backing for carpets, semirigid foams, rigid insulation foams, cellular elastomers, compact elastomers, thermoplastic polyurethanes, fiber applications, and surface-coating applications.

One raw material for preparing the polyurethane is the isocyanate. Examples of isocyanates are MDI, TDI, and HDI. Depending on the number of the rings, a distinction can also be made between, by way of example, monomeric MDI having 2 isocyanate groups, for example 4,4′-MDI, and polymeric MDI. Polynuclear or higher-functionality isocyanates can also be prepared from monomeric isocyanates via uretdione formation or isocyanurate formation or biuret formation.

Organic isocyanates tend to discolor while in storage. In particular at relatively high temperatures, e.g. in a feed vessel of a processing machine, this discoloration can lead to a considerable reduction in product quality. The prior art therefore stabilizes isocyanates.

Known stabilizers for stabilizing isocyanates are sterically hindered phenols, aromatic amines, thio compounds, phenothiazines, phosphites, and mixtures of these products. The compound most often used for stabilization is 2,6-di-tert-butyl4-methylphenol (BHT).

However, isocyanates are merely precursors for the actual polyurethane product, which, as described above, can be used for a variety of applications. Incorrect selection of the stabilizer can sometimes have an adverse effect on the properties of the final product. For example, aromatic amines tend to discolor significantly when they are exposed to sunlight. A polyurethane film produced from an isocyanate stabilized with an aromatic amine therefore has only restricted suitability for use in direct sunlight. BHT, in turn, is a stabilizer with high volatility. The material can therefore sometimes volatilize before processing of the isocyanate to give the finished polyurethane is complete. This can lead to environmental problems or else problems with health and safety at the workplace. In addition, the BHT can migrate from the finished product subsequently and evaporate. By way of example, in the automotive industry this can lead to exceeding of the prescribed values for evaporation of volatiles from polyurethane.

WO 99/48863 therefore proposes the use of 3,5-di-tert-butyl-4-hydroxyphenylpropionic esters for stabilizing isocyanates.

However, some 3,5-di-tert-butyl-4-hydroxyphenylpropionic esters are unsuitable for stabilizing isocyanates. For example, many 3,5-di-tert-butyl-4-hydroxyphenylpropionic esters are highly crystalline with a high melting point. Examples of these compounds are the products marketed by Ciba Specialty Chemicals with the trademarks Irganox® 1330, Irganox® 1010, and Irganox® 1098. These products cannot be incorporated into the isocyanate at the temperatures usual in the processing of isocyanates. Other 3,5-di-tert-butyl-4-hydroxyphenylpropionic esters, in turn, have higher molar mass than BHT, but the molar mass is still inadequate to comply with the demanding values applicable to fogging and volatility in some applications. Examples of these compounds are methyl 3,5-di-tert-butyl-4-hydroxyphenylpropionate and Irganox® 1135.

It is an object of the present invention, therefore, to develop mixtures (1) comprising isocyanate and stabilizer, i.e. to develop a stabilizer which is intended for isocyanates and which protects the isocyanates from discoloration during storage and heating, and is easy to incorporate, and has minimum volatility, and also does not constrain the properties of the polyurethane end product or, therefore, its use.

We have found that this object is achieved by way of the mixtures described at the outset.

Preference is given to mixtures (1) comprising (a) isocyanate and (b) preferably amorphous or liquid stabilizers comprising at least two phenolic groups bonded to one another by way of, as bonding radical (II), a polyol with a number-average molar mass of from 40×F to 1000×F g/mol, preferably from 75×F to 500×F g/mol, in particular from 90×F to 150×F g/mol, where F is the number of phenolic groups in the molecule. In order to calculate the molar mass range, therefore, the number of phenolic groups is multiplied by the appropriate factor, for example by 40 and 1000. If the number of phenolic groups is two (F=2), therefore, the resultant molar mass range is from 80 to 2000 g/mol. The bonding radical (II) preferred according to the invention therefore preferably has a number-average molar mass of from 40×F to 1000×F g/mol, preferably from 75×F to 500×F g/mol, in particular from 90×F to 150×F g/mol, where F is the number of phenolic active ingredient groups (I). This molar mass relates to (II). Preference is given to stabilizers where F=2, i.e. those having two phenolic groups.

The stabilizers therefore preferably comprise two structural units. Firstly, at least two phenolic active ingredient groups (I), bonded to one another via a polyol with compatibilizing and amorphizing action, for example a polyether, polyester, polycarbonatediol, polythioether, and/or polyether polythioether. The polyether, polyester, polycarbonatediol, polythioether, and/or polyether polythioether is the bonding radical (II). The bonding of the phenolic groups (I) to the bonding radical (II) may, by way of example, be brought about by way of ester groups, amide groups, and/or thioester groups, preferably ester groups. By way of example, the inventive stabilizers may therefore be prepared by well-known esterification and/or amidation of active ingredients which have at least one phenolic group, and also at least one carboxy group, with polyethers, with polycarbonatediols, with polyesters, with polythioethers, and/or with polyether polythioethers, where these have at least two free groups reactive toward carboxy groups, examples of these groups being hydroxy groups and/or amino groups. Surprisingly, it has been found here that the color of the stabilizers is particularly good when a reducing agent is present during the synthesis, this preferably being a phosphorus compound, in particular a compound of trivalent phosphorus. Examples of suitable phosphorus compounds may be found in Plastics Additive Handbook, 5th edition, H. Zweifel, eds., Hanser Publishers, Munich, 2001 ([1]), pp. 109-112.

By way of example, the following groups

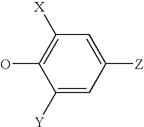

may be present as active ingredient groups (I)

where

X and Y, independently of one another, are hydrogen or straight-chain, branched-chain, or cyclic alkyl groups having from 1 to 12 carbon atoms,

Z is at least one carboxy group bonded by way of an alkylene radical to the phenol radical.

The parent groups used preferably comprise the following compounds:

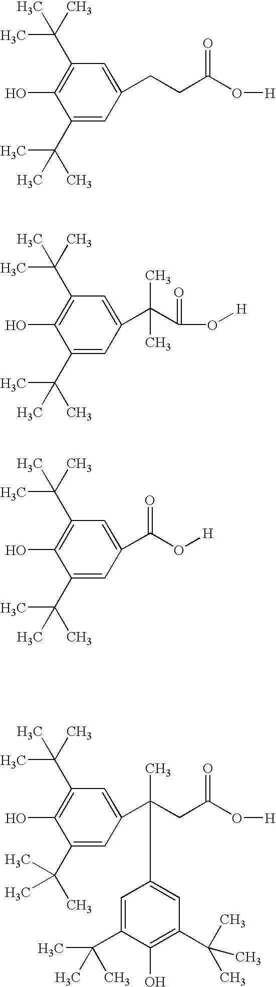

The following compound is particularly preferred as (I)

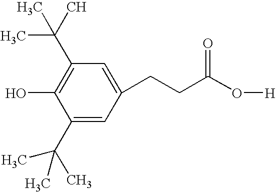

The radical (I) may be used in the form of anhydride, acid chloride, ester, or free acid for bonding to the bonding radical (II). The radical Z in the formulae above varies correspondingly. According to the invention, these phenolic groups (I) are bonded to one another by way of the carboxy group of (I) through a bonding radical (II).

The ratio by weight of compatibilizing radical (II) to the active ingredient (I) is optimized through the preferred molar mass (II). If appropriate, nitrogen or oxygen involved in the bonding of (II) to (I) in the amide or ester structure is counted when determining the molar mass of (II).

Preference is therefore given to stabilizers (b), also termed antioxidants in this specification, where the phenolic active ingredient groups (I) have bonding via a bonding radical (II). Preference is given here to bonding radicals (II) which have different molar masses, i.e. where the number-average molar mass is smaller than the weight-average molar mass (Mn<Mw). This molar mass distribution suppresses any undesired crystallization of the stabilizers.

By way of example, the bonding radical (II) used may comprise well-known polyols, such as polyesters, polycarbonatediols, polyethers, polythioethers, and/or polyether polythioethers, preferably polyethers, where these have at least two groups reactive toward carboxy groups, for example hydroxy groups, thiol groups, and/or amino groups, such as primary amino groups, where these can be reacted with carboxy groups of (I) to prepare the inventive stabilizer. The structure of the bonding radical (II) may be linear or branched.

By way of example, the stabilizers (b) may have the following general structure:
(I)—X—R—[Y—R]n-X—(I),

or, if the stabilizer has functionality >2,
[(I)—X—R—[Y—R]n-Y]z-A

where

(I) is the active ingredient group described at the outset, bonded by way of its carboxy group,

X is —O—, —S—, or —NH—, preferably —O—

Y is —O— or —S—, preferably —O—

R is C₂-C₁₂-alkyl, which may be straight-chain or branched

n is a whole number which achieves the inventive molar mass

A is a hydrocarbon skeleton having from 3 to 20 carbon atoms

z is 3, 4, 5, 6, 7, or 8

and where, if X, Y, and R occur more than once in (II) they may in each case have meanings which differ and are independent of one another. By way of example, X may mean both sulfur and oxygen within one bonding radical (II). The definition of n applies to all of the formulae arising in this specification.

The following radicals may preferably be used as bonding radical (II):

Polytetrahydrofuran with a number-average molar mass of from 200 to 300 g/mol, and

Polyethylene glycol with a number-average molar mass of from 150 to 300 g/mol.

It may moreover be advantageous to mix various radicals (II), and to react the mixture with (I) in order to adjust the viscosity, compatibility, solubility, and bloom performance of the resultant stabilizer to ideal values.

Particular preference is given to mixtures (1) comprising the following phenolic stabilizer (b):

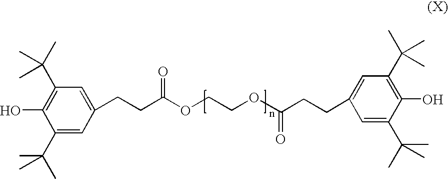

where n is a whole number in the range from 1 to 31, preferably 2, 3, 4, 5, or 6, particularly preferably 3 or 4. n is in particular selected in such a way that the number-average molar mass of the stabilizer is from 700 to 800 g/mol. n is particularly preferably selected in such a way that in the aggregate, i.e. in the stabilizer mixture comprising the individual stabilizer molecules, the weight-average molar mass of the stabilizer mixture is greater than the number-average molar mass of the stabilizer mixture.

Particular preference is also given to mixtures (1) comprising the following phenolic stabilizer (b):

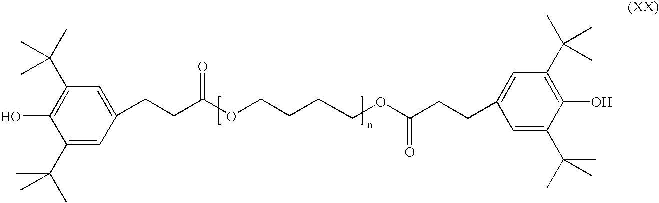

where n is a whole number in the range from 1 to 31, preferably 2, 3, 4, 5, or 6, particularly preferably 3 or 4. n is in particular selected in such a way that the number-average molar mass of the stabilizer is from 700 to 900 g/mol. n is particularly preferably selected in such a way that in the aggregate, i.e. in the stabilizer mixture comprising the individual stabilizer molecules, the weight-average molar mass of the stabilizer mixture is greater than the number-average molar mass of the stabilizer mixture. This means that the preferred antioxidants (X) and (XX) are particularly preferably used in mixtures composed of various compounds of the formula (X) and/or (XX) which differ in the values of n. The proportion of the molecules having n=1, n=2, n=3, etc. up to n=31 here is preferably selected in such a way that the number-average molar mass of the antioxidant mixture corresponds to the molar mass recognized as advantageous. The proportion of the molecules having n=1, n=2, n=3, up to n=31 is preferably selected in such a way that the number-average molar mass of the antioxidant mixture, i.e. of the inventive stabilizer (b), is from 600 to 10000 g/mol, preferably from 700 to 10000 g/mol, particularly preferably from 700 to 3000 g/mol, in particular from 700 to 900 g/mol. In another preferred embodiment, use is made of antioxidant mixtures whose polydispersity P_{d} is greater than 1, meaning that their number-average molar mass is smaller than their weight-average molar mass. By way of example, this condition is complied with if the antioxidant is composed of a mixture composed of various molecules of the structure (X) or (XX) with different values of n.

The advantage of a liquid stabilizer is that liquid metering is easier than solids metering. This presupposes that the finished stabilizer has a certain viscosity.

Radicals (II) which are suitable for easy incorporation are those where the condensation product of (I) and (II) has a viscosity at room temperature (25° C.) of η=from 10⁻² to 10² Pas, preferably η=from 10⁻¹ to 10¹ Pas. The amount of the stabilizers (b) present in the inventive mixtures (1) is preferably from 1 to 50000 ppm, preferably from 100 to 10000 ppm, particularly preferably from 200 to 1500 ppm, in particular from 250 to 750 ppm, based in each case on the total weight of the mixture (1) comprising isocyanate and stabilizer. In addition to the inventive stabilizers, other well-known stabilizers may be used in the mixtures, examples being phosphites, thiosynergists, HALS compounds, UV absorbers, quenchers, and sterically hindered phenols.

Preferred organic isocyanates (a) which may be used are well-known aliphatic, cycloaliphatic, araliphatic, and/or aromatic isocyanates, such as tri-, tetra-, penta-, hexa-, hepta-, and/or octamethylene diisocyanate, 2-methylpentamethylene 1,5-diisocyanate, 2-ethylbutylene 1,4-diisocyanate, pentamethylene 1,5-diisocyanate, butylene 1,4-diisocyanate, 1-isocyanato-3,3,5-trimethyl-5-isocyanatomethylcyclohexane (isophorone diisocyanate, IPDI), 1,4- and/or 1,3-bis-(isocyanatomethyl)cyclohexane (HXDI), cyclohexane 1,4-diisocyanate, 1-methylcyclohexane 2,4- and/or 2,6-diisocyanate, and/or dicyclohexylmethane 4,4′-, 2,4′- and 2,2′-diisocyanate, diphenylmethane 2,2′-, 2,4′- and/or 4,4′-diisocyanate (MDI), naphthylene 1,5-diisocyanate (NDI), tolylene 2,4- and/or 2,6-diisocyanate (TDI), polymeric MDI, diphenylmethane diisocyanate, 3,3′-dimethyldiphenyl diisocyanate, 1,2-diphenylethane diisocyanate, and/or phenylene diisocyanate. The isocyanates present in the inventive mixture (1) may, where appropriate, be in modified form, e.g. in the form of biuretes, allophanates, and/or urethane, and/or may have urea structures. Preferred isocyanates (a) are diphenylmethane 2,2′-, 2,4′- and/or 4,4′-diisocyanate (MDI), naphthylene 1,5-diisocyanate (NDI), and/or tolylene 2,4- and/or 2,6-diisocyanate (TDI).

All of the molar masses mentioned in this specification have the unit [g/mol].

The inventive mixtures (1) are preferably used in processes for preparing polyurethanes. The preparation of polyurethanes, if appropriate having urea structures, biuret structures, allophanate structures, and/or isocyanurate structures, is well-known and usually involves reaction of (a) isocyanates with (b) compounds reactive toward isocyanates and having a molar mass of from 500 to 10000, if appropriate in the presence of (d) catalysts, (e) blowing agents, and/or (f) conventional auxiliaries and/or conventional additives. According to the invention, the isocyanate component used for reaction with compounds reactive toward isocyanate comprises the inventive mixture (1), this preferably being the only isocyanate component, or else, if appropriate, together with other isocyanates. Examples of polyurethane products are flexible foam applications, such as mattresses, foam backing for carpets, semirigid foams, rigid insulation foams, cellular elastomers, compact elastomers, thermoplastic polyurethanes, and fiber applications and surface-coating applications. The production of these products has been widely described.

The advantages of the invention will be described using the examples below.

### EXAMPLES

### Example 1

### Preparation of an Inventive Stabilizer

110 g of polytetrahydrofuran (PTHF 250) (molar mass 226.85 g/mol; 0.4849 mol) were placed in a 500 ml flask with 277.9 g of methyl 3-(3,5-di-tert-butyl-4-hydroxyphenyl)propionate (molar mass 292.4 g/mol; 0.9504 mol), and also 1000 ppm of potassium methoxide. The solution was flushed with nitrogen and then heated to 140° C., with stirring. Passing of nitrogen through the solution was continued during the reaction. The resultant methanol was removed by freezing in a cold trap (liquid nitrogen). The reaction was terminated after 7 h. GPC analysis showed complete conversion of the methyl 3-(3,5-di-tert-butyl-4-hydroxyphenyl)propionate. To remove the potassium methoxide, phosphoric acid (85% of the stoichiometric amount of the potassium methoxide) was added at 80° C. After stirring for 30 min, 3% by weight of water were added at 80° C., the associated stirring time being 2 h. The excess water was then removed by distillation, and the precipitated salt was removed by filtration.

### Example 2

### Preparation of an Inventive Stabilizer

25 g of polyethylene glycol (Pluriol® E.200, BASF Aktiengesellschaft) (OHZ 557 mg of KOH/g) were weighed into a four-necked flask with 71.13 g of methyl 3-(3,5-di-tert-butyl-4-hydroxyphenyl)propionate (Raschig), and heated to 145° C. A stream of nitrogen was continuously passed through the solution during the heating phase and the transesterification reaction. Once 145° C. had been reached, 0.177 g=2000 ppm of potassium methoxide were added to the solution in order to initiate the actual trans-esterification reaction. The resultant methanol was removed by freezing in a down-stream cold trap (liquid nitrogen). The product was cooled to 80° C. after 6 h of reaction time. To neutralize the product, 0.246 g of 85% strength phosphoric acid was then added to the flask at 80° C. Stirring of the product was continued for half an hour, and the mixture was then filtered through a SeitzSchenk pressure filter, using a T750 filter (retention level from 4 to 10 μm). The conversion in the transesterification reaction, determined via gel permeation chromatography, was more than 95% in all of the examples. The potassium content was determined by atomic absorption spectroscopy and was less than 20 ppm of potassium in all of the experiments.

### Example 3

### Volatility Studies

The volatility of Irganox® 1135 and of the inventive stabilizer from Example 1 were studied thermogravimetrically. For this, the two products were heated, using a heating rate of 2.5 K/min, and the weight loss of the specimen was recorded. The inventive stabilizer showed significantly less volatility.

### Example 4

### Solubility in Isocyanates

1000 ppm of Irganox® 1330 were added to 4,4′ MDI, and the mixture was stored at 45° C. in a heating cabinet. A sediment of the stabilizer can still be seen even after 12 h. In contrast, the inventive stabilizer from Examples 1 and 2 forms a homogeneous mixture with the MDI in a very short time.

### Example 5

### Stabilization of Isocyanates

Lupranat® ME (BASF Aktiengesellschaft) was stabilized with the commercially available phenolic stabilizers Irganox® 1076, and Irganox® 1141. An experiment with the inventive stabilizer from Example 2 for stabilization was used as comparator. The isocyanate was stored for up to 8 weeks at 42° C. To determine the discoloration of the isocyanate, as a measure of stabilization quality, the alpha value was measured. Table 1 states the alpha value as a function of time for the various stabilizers, and shows that, even when its concentration is lower, the inventive stabilizer gives excellent performance in protecting the MDI.

TABLE 1

| | | | 600 ppm of |
|---|---|---|---|
| Time | 1000 ppm | 600 ppm | stabilizer from |
| in weeks | Irganox ® 1076 | Irganox ® 1141 | Example 2 |
| 0 | 18 | 53 | 18 |
| 1 | 13 | 51 | 13 |
| 2 | 17 | 113 | 16 |
| 3 | 22 | 264 | 17 |
| 4 | 33 | 613 | 19 |
| 6 | 82 | | 30 |

### Example 6

155 g of a trifunctional polyetherol (BASF Aktiengesellschaft) and 200 g of methyl 3-(3,5-di-tert-butyl4-hydroxyphenyl)propionate were placed in a 500 ml flask. The mixture was heated to 100° C. with nitrogen flushing. 35 mg of titanium tetrabutoxide were then added. The mixture was heated to 165° C., with stirring and continued nitrogen flushing, and the pressure was slightly reduced. After 2 h at 165° C., the mixture was heated to 170° C., and after 2 further hours was heated to 175° C. After 4 further hours the reaction was terminated.

### Example 7

122.6 g of methyl 3-(3,5-di-tert-butyl-4-hydroxyphenyl)propionate (420 mmol), 48.3 g of polyetheramine D 230 (BASF Aktiengesellschaft), 2 g of p-toluenesulfonic acid (10 mmol), and 0.5 g of 50% strength hypophosphorous acid were heated to 180° C. in a 500 ml flask. The resultant methanol was removed via a distillation bridge. The conversion was determined via the amine number and was 91% after 4 h. The product was pale yellow, glassy, and clear.

1. A mixture comprising:
(a) isocyanate; and
(b) stabilizers with a molar mass of from 600 to 10000 g/mol that comprise at least two phenolic groups.

2. A mixture comprising:
(a) isocyanate; and
(b) stabilizers that comprise at least two phenolic groups bonded to one another by way of, as a bonding radical (II), a polyol with a number-average molecular weight of from 40×F to 1000×F g/mol, wherein F is the number of phenolic groups in the molecule.

3. The mixture according to claim 1, wherein, in the stabilizer (b), the phenolic groups are active ingredient groups (I) bonded by way of a bonding radical (II).

4. The mixture according to claim 1, wherein a number-average molecular weight (Mn) of the bonding radical (II) is less than a weight-average molecular weight (Mw) of the bonding radical (II).

5. A mixture comprising;
(a) isocyanate;
as stabilizer (b), 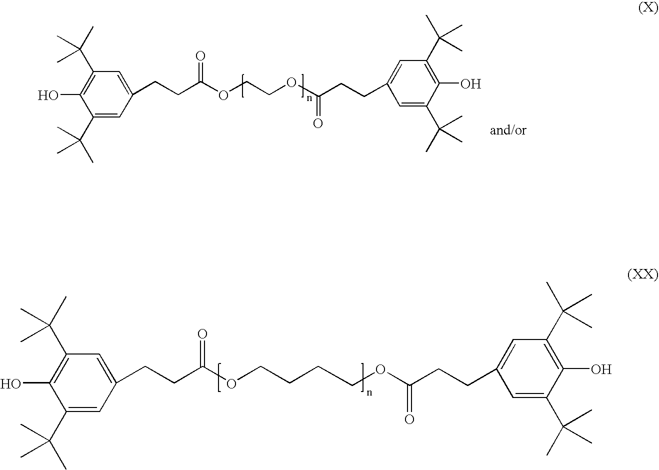
wherein each n is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31.

6. The mixture according to claim 1, wherein an amount present of the stabilizer (b) in the mixture that comprises isocyanate and the stabilizer is from 1 ppm to 50000 ppm, based on a total weight of the mixture.

7. The mixture according to claim 1, wherein the isocyanate (a) is at least one of:
diphenylmethane 2,2′-diisocyanate (MDI), diphenylmethane 2,4′-diisocyanate (MDI), dihenylmethane 4,4′-diisocyanate (MDI), naphthylene 1,5-diisocyanate (NDI). tolylene 2,4-diisocyanate (TDI), and tolylene 2,6-diisocyanate (TDI).

8. A process for preparing polyurethanes, comprising:
reacting the mixture of claim 1 to obtain a polyurethane.

9. The mixture of claim 2, wherein the number-average molecular weight of the polyol is from 75×F to 500×F g/mol, wherein F is the number of phenolic groups in the molecule.

10. The mixture of claim 2, wherein the number-average molecular weight of the polyol is from 90×F to 150×F g/mol, wherein F is the number of phenolic groups in the molecule.

11. The mixture of claim 2, wherein, in the stabilizer (b), the phenolic groups are active ingredient groups (I) bonded by way of a bonding radical (II).

12. The mixture of claim 11, wherein a number-average molecular weight (Mn) of the bonding radical (II) is less than a weight-average molecular weight (Mw) of the bonding radical (II).

13. The mixture of claim 2, wherein an amount present of the stabilizer (b) in the mixture that comprises isocyanate and the stabilizer is from 1 ppm to 50000 ppm, based on a total weight of the mixture.

14. The mixture of claim 5, wherein an amount present of the stabilizer (b) in the mixture that comprises isocyanate and the stabilizer is from 1 ppm to 50000 ppm, based on a total weight of the mixture.

15. The mixture of claim 2, wherein the isocyanate (a) is at least one of:
diphenylmethane 2,2′-diisocyanate (MDI); diphenylmethane 2,4′-diisocyanate (MDI), diphenylmethane 4,4′-diisocyanate (MDI); naphthylene 1,5-diisocyanate (NDI); tolylene 2,4-diisocyanate (TDI), and tolylene 2,6-diisocyanate (TDI).

16. The mixture of claim 5, wherein the isocyanate (a) is at least one of:
diphenylmethane 2,2′-diisocyanate (MDI); diphenylmethane 2,4′-diisocyanate (MDI), diphenylmethane 4,4′-diisocyanate (MDI); naphthylene 1,5-diisocyanate (NDI); tolylene 2,4-diisocyanate (TDI), and tolylene 2,6-diisocyanate (TDI).

17. A process for preparing polyurethanes, comprising:
reacting the mixture of claim 2 to obtain a polyurethane.

18. A process for preparing polyurethanes, comprising:
reacting the mixture of claim 5 to obtain a polyurethane.
